(19) United States
(12) Patent Application Publication
(10) Pub. No.: US 2009/0093718 A1
(43) Pub. Date: Apr. 9, 2009

(54) ULTRASONIC DIAGNOSIS APPARATUS, RADIOFREQUENCY WAVE CAUTERY TREATMENT DEVICE, ULTRASONIC DIAGNOSIS AND TREATMENT SYSTEM, AND ULTRASONIC DIAGNOSIS AND TREATMENT APPARATUS

(76) Inventors: Takao Jibiki, Tokyo (JP); Masaki Tsuda, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/329,037

(22) Filed: Dec. 5, 2008

(30) Foreign Application Priority Data
Jul. 12, 2007 (JP) 2007-316675

(51) Int. Cl. A61B 8/14

(52) U.S. Cl. 600/443

(57) ABSTRACT

An ultrasonic diagnosis and treatment system includes an ultrasonic probe for transmitting and receiving the ultrasonic waves, an image generator unit for generating an ultrasonic image based on the echo signals received by the ultrasonic probe, and a biopsy needle for radiating the radiofrequency waves. The ultrasonic diagnosis and treatment system includes a controller unit for alternately performing the transmission and reception of the ultrasonic wave by means of the ultrasonic probe and the radiofrequency wave radiation by the biopsy needle.

### CROSS REFERENCE TO RELATED APPLICATIONS

This application claims the benefit of Japanese Patent Application No. 2007-316675 filed Dec. 7, 2007, which is hereby incorporated by reference in its entirety.

### BACKGROUND OF THE INVENTION

The embodiments described herein are is related to an ultrasonic diagnosis apparatus, a radiofrequency wave cautery treatment device, an ultrasonic diagnosis and treatment system which is capable of performing the cautery treatment by using the radiofrequency wave, and an ultrasonic diagnosis and treatment apparatus.

In an ultrasonic diagnosis and treatment system which is capable of performing the cautery treatment by using the radiofrequency wave, a biopsy needle is inserted into a subject while confirming on an ultrasonic image to conduct the cautery treatment of for example a tumor by radiating radiofrequency waves from the biopsy needle (see for example Japanese Unexamined Patent Publication No. 2007-135988). In such an ultrasonic diagnosis and treatment system, the biopsy needle is inserted while viewing an ultrasonic image on the real time basis, so that the transmission and the reception of the ultrasonic waves from an ultrasonic probe for generating the ultrasonic image is performed at the same time as the radiation of radiofrequency waves from the biopsy needle.

In such a case the frequency of radiofrequency waves may be set to approximately 450 kHz. The harmonic waves of the radiofrequency waves at that frequency may be overlapped with the frequency band of the ultrasonic waves to be transmitted and to be received by using the ultrasonic probe (approximately a few MHz) to cause some noises, which may appear on the ultrasonic image, resulting in the problem of the degradation of the image quality.

### BRIEF DESCRIPTION OF THE INVENTION

It is desirable that the problem described previously is solved.

A first aspect provides an ultrasonic diagnosis apparatus having an ultrasonic probe for transmitting and receiving ultrasonic waves; and an image generator unit for generating an ultrasonic image based on echo signals received by the ultrasonic probe; the ultrasonic diagnosis apparatus including a controller unit for alternatively instructing the ultrasonic probe to transmit and receive ultrasonic waves and a radiofrequency wave cautery treatment device having a biopsy needle for radiating radiofrequency waves to radiate radiofrequency waves.

A second aspect provides, an ultrasonic diagnosis apparatus in accordance with the first aspect described above, in which: the controller unit includes a transmission instructor unit for outputting the transmission instruction signal to transmitter unit which provides to the ultrasonic probe driving signal to transmit ultrasonic waves, and a permission signal generator unit for generating radiofrequency wave radiation permission signal for permitting the radiation of the radiofrequency waves from the biopsy needle; the permission signal generator unit outputs the radiofrequency wave radiation permission signal to the radiofrequency wave radiation permission unit of the radiofrequency wave cautery treatment device which has a generator unit of a radiofrequency wave radiation instruction signal for radiating the radiofrequency waves from the biopsy needle, and a radiofrequency wave radiation permission unit for radiating the radiofrequency waves from the biopsy needle by outputting the radiofrequency wave radiation instruction signal only when the radiofrequency wave radiation instruction signal is input from the radiofrequency wave radiation instruction signal generator unit and if the radiofrequency wave radiation permission signal is input from the permission signal generator unit; and the output of the transmission instruction signal from the transmission instruction unit and the output of the radiofrequency wave radiation permission signal from the permission signal generator unit are alternately performed.

A third aspect provides a radiofrequency wave cautery treatment device including a biopsy needle for radiating radiofrequency waves, wherein the radiation of the radiofrequency waves from the biopsy needle is controlled by the controller unit of the ultrasonic diagnosis apparatus set forth in the first aspect described above.

A fourth aspect provides a radiofrequency wave cautery treatment device in accordance with the third aspect described above, including: a generator unit of the radiofrequency wave radiation instruction signal for radiating the radiofrequency waves from the biopsy needle; and a radiofrequency wave radiation permission unit for outputting the radiofrequency wave radiation instruction signal to radiate the radiofrequency waves from the biopsy needle only when the radiofrequency wave radiation instruction signal is input from the radiofrequency wave radiation instruction signal generator unit and if the radiofrequency wave radiation permission signal is input from the permission signal generator unit in the ultrasonic diagnosis apparatus set forth in the second aspect described above.

A fifth aspect provides an ultrasonic diagnosis and treatment system, including: an ultrasonic probe for transmitting and receiving the ultrasonic waves; an image generator unit for generating an ultrasonic image based on the echo signals received by the ultrasonic probe; and a biopsy needle for radiating the radiofrequency waves, the ultrasonic diagnosis and treatment system including: a controller unit for alternately performing the transmission and reception of the ultrasonic wave by means of the ultrasonic probe and the radiofrequency wave radiation by the biopsy needle.

A sixth aspect provides an ultrasonic diagnosis and treatment system in accordance with the fifth aspect described above, including: a body of a radiofrequency wave cautery treatment device having the biopsy needle connected thereto, wherein: the controller unit includes a transmission instruction unit for outputting the transmission instruction signal to the transmitter unit for providing the driving signal to the ultrasonic probe for transmitting the ultrasonic waves therefrom, and a permission signal generator unit for generating radiofrequency wave radiation permission signal for permitting the radiation of the radiofrequency waves from the biopsy needle, the output of the transmission instruction signal from the transmission instruction unit and the output of the radiofrequency wave radiation permission signal from the permission signal generator unit being alternately performed; and the body of the radiofrequency wave cautery treatment device includes a generator unit for radiofrequency wave radiation instruction signal for radiating the radiofrequency waves from the biopsy needle, and a radiofrequency wave radiation permission unit for outputting the radiofrequency wave radiation instruction signal to permit the biopsy needle to radiate the radiofrequency waves only when the radiofrequency wave radiation instruction signal is input from the radiofrequency wave radiation instruction signal generator unit and if the radiofrequency wave radiation permission signal is input from the permission signal generator unit.

A seventh aspect provides an ultrasonic diagnosis and treatment system in accordance with the sixth aspect described above, wherein the radiofrequency wave radiation instruction signal from the radiofrequency wave radiation permission unit is output to a voltage generator unit for applying to the biopsy needle the voltage for radiating the radiofrequency waves.

An eighth aspect provides an ultrasonic diagnosis and treatment system in accordance with the fifth aspect described above, which includes: an ultrasonic diagnosis apparatus including the ultrasonic probe, the image generator unit, and the controller unit; and a radiofrequency wave cautery treatment device including the biopsy needle.

A ninth aspect provides an ultrasonic diagnosis and treatment apparatus, including: a body having an ultrasonic probe for transmitting and receiving the ultrasonic waves and a biopsy needle for radiating the radiofrequency wave connected thereto, the body including an image generator unit for generating an ultrasonic image based on the echo signals received by the ultrasonic probe, wherein: the body includes a controller unit for alternately performing the transmission and the reception of the ultrasonic waves by means of the ultrasonic probe and the radiofrequency wave radiation by the biopsy needle.

A tenth aspect provides an ultrasonic diagnosis and treatment apparatus in accordance with the ninth aspect described above, wherein the controller unit has: a transmission instruction unit outputting the transmission instruction signal to the transmitter unit for providing the driving signal to the ultrasonic probe to transmit the ultrasonic waves; and a generator unit of the radiofrequency wave radiation instruction signal for radiating the radiofrequency waves from the biopsy needle, and the output of the transmission instruction signal from the transmission instruction unit and the output of the radiofrequency wave radiation instruction signal from the radiofrequency wave radiation instruction signal generator unit are alternately performed.

An eleventh aspect provides an ultrasonic diagnosis and treatment apparatus in accordance with the ninth aspect described above, wherein: the controller unit has: a transmission instruction unit for outputting the transmission instruction signal to the transmitter unit for providing the driving signal to the ultrasonic probe to transmit the ultrasonic waves; and a permission signal generator unit for outputting the radiofrequency wave radiation permission signal for permitting to radiate the radiofrequency wave from the biopsy needle, and the output of the transmission instruction signal from the transmission instruction unit and the output of the radiofrequency wave radiation permission signal from the permission signal generator unit are alternately performed; and the body has: a generator unit of the radiofrequency wave radiation instruction signal for radiating the radiofrequency waves from the biopsy needle; and the radiofrequency wave radiation permission unit for outputting the radiofrequency wave radiation instruction signal to permit the biopsy needle to radiate the radiofrequency waves only when the radiofrequency wave radiation instruction signal is input from the radiofrequency wave radiation instruction signal generator unit and if the radiofrequency wave radiation permission signal from the permission signal generator unit is input.

A twelfth aspect provides an ultrasonic diagnosis apparatus, including: an ultrasonic probe for transmitting and receiving ultrasonic waves; a transmission controller unit for controlling the transmission of the ultrasonic waves from the ultrasonic probe; and an image generator unit for generating an ultrasonic image based on the echo signals received by the ultrasonic probe, wherein: the transmission controller unit receives the information with respect to the radiofrequency wave radiation from the radiofrequency wave cautery treatment device having a biopsy needle for radiating the radiofrequency waves, and the transmission of ultrasonic waves from the ultrasonic probe is performed when the radiofrequency waves are not radiated from the biopsy needle.

A thirteenth aspect provides a radiofrequency wave cautery treatment device having a biopsy needle for radiating the radiofrequency wave, the radiofrequency wave cautery treatment device outputting information with respect to the radiofrequency wave radiation to the ultrasonic diagnosis apparatus in accordance with the twelfth aspect described above.

A fourteenth aspect provides an ultrasonic diagnosis and treatment system, including: an ultrasonic probe for transmitting and receiving ultrasonic waves; a transmission controller unit for controlling the transmission of the ultrasonic waves from the ultrasonic probe; an image generator unit for generating an ultrasonic image based on the echo signals received by the ultrasonic probe; a biopsy needle for radiating the radiofrequency waves; and a radiation controller unit for controlling the radiation of the radiofrequency waves from the biopsy needle; wherein: the transmission controller unit receives information with respect to the radiofrequency wave radiation from the radiation controller unit, and the ultrasonic waves transmission from the ultrasonic probe is performed when the radiofrequency wave is not radiated from the biopsy needle.

A fifteenth aspect provides an ultrasonic diagnosis and treatment system in accordance with the fourteenth aspect described above, including: a ultrasonic diagnosis apparatus having the ultrasonic probe, the transmission controller unit, and the image generator unit; and a radiofrequency wave cautery treatment device having the biopsy needle and the radiation controller unit.

In accordance with the first aspect described above, the controller unit instructs alternately the transmission and the reception of the ultrasonic waves by the ultrasonic probe, and the radiation of the ultrasonic waves from the radiofrequency wave cautery treatment device, so that the radiofrequency waves are not radiated when the transmission and the reception of ultrasonic waves by using the ultrasonic probe are performed. Therefore the noises that are caused by the radiofrequency waves will not appear in the obtained ultrasonic image, allowing preventing the degradation of the image quality.

In accordance with the second aspect described above, in the radiofrequency wave cautery treatment device, the radiofrequency wave radiation permission unit outputs the radiofrequency wave radiation instruction signal to allow the radiation of the radiofrequency waves from the biopsy needle only when the radiofrequency wave radiation instruction signal is input and if the radiofrequency wave radiation permission signal is also input. And in the ultrasonic diagnosis apparatus, since the output of the transmission instruction of the ultrasonic waves from the transmission instruction unit and the output of the radiofrequency wave radiation permission signal from the permission signal generator unit are alternately performed, the radiofrequency wave radiation permission signal may not be input to the radiofrequency wave radiation permission unit at the time of the transmission and the reception of the ultrasonic waves, so that the transmission and the reception of the ultrasonic waves by using the ultrasonic probe and the radiation of the radiofrequency wave from the biopsy needle will be alternately performed.

In accordance with the third aspect described above, when the transmission and the reception of the ultrasonic waves by using the ultrasonic probe are performed, the radiofrequency waves are not radiated from the biopsy needle so that the noises which are caused by the radiofrequency waves will not appear in the ultrasonic image that is obtained in the ultrasonic diagnosis apparatus, allowing preventing the degradation of the image quality.

In accordance with the fourth aspect described above, the radiofrequency wave radiation permission unit outputs the radiofrequency wave radiation instruction signal to radiate the radiofrequency waves from the biopsy needle only when the radiofrequency wave radiation instruction signal is input and if the radiofrequency wave radiation permission signal is also input. And in the ultrasonic diagnosis apparatus, since the output of the transmission instruction of the ultrasonic waves from the transmission instruction unit and the output of the radiofrequency wave radiation permission signal from the permission signal generator unit are alternately performed, the radiofrequency wave radiation permission signal will not be input to the radiofrequency wave radiation permission unit at the time of the transmission and the reception of the ultrasonic waves. Therefore when performing the transmission and the reception of the ultrasonic waves, the radiofrequency waves will not be radiated from the biopsy needle, so that the noises which are caused by the radiofrequency waves will not appear in the ultrasonic image obtained by the ultrasonic diagnosis apparatus, allowing preventing the degradation of the image quality.

In accordance with the fifth aspect described above since the controller unit instructs to perform alternately the transmission and the reception of the ultrasonic waves by using the ultrasonic probe and the radiation of the radiofrequency waves from the radiofrequency wave cautery treatment device, the radiofrequency waves will not be radiated at the time when the transmission and the reception of the ultrasonic waves by using the ultrasonic probe are performed, so that the noises which are caused by the radiofrequency waves will not appear in the obtained ultrasonic image, allowing preventing the degradation of the image quality.

In accordance with the sixth aspect described above, the radiofrequency wave radiation permission unit outputs the radiofrequency wave radiation instruction signal to allow to radiate the radiofrequency waves from the biopsy needle, only when the radiofrequency wave radiation instruction signal is input and the radiofrequency wave radiation permission signal is also input.

In accordance with the seventh aspect described above, the radiofrequency wave radiation instruction signal is input to the voltage generator unit to apply the voltage to the biopsy needle, and the transmission and the reception of the ultrasonic waves and the radiation of the radiofrequency waves are alternately performed.

In accordance with the eighth aspect described above in a ultrasonic diagnosis and treatment system including: the ultrasonic diagnosis apparatus having the ultrasonic probe, the image generator unit and the controller unit; and the radiofrequency wave cautery treatment device having the biopsy needle, the degradation of the image quality of the ultrasonic image which are caused by the radiofrequency waves may be prevented.

In accordance with the ninth, tenth, and eleventh aspects described above, in addition to the same effect as the first and the fifth aspects described above, a function of generating the ultrasonic image by the transmission and the reception of the ultrasonic waves, and a function of performing the cautery treatment by radiating the radiofrequency waves are integrated as one apparatus, the control associating the control of the generation of the ultrasonic image with the control of the radiation of the radiofrequency waves may be achieved with a simple configuration.

In accordance with the twelfth aspect described above since the transmission and the reception of the ultrasonic waves by using the ultrasonic probe are performed only when the radiofrequency waves are not radiated from the biopsy needle in the radiofrequency wave cautery treatment device, the noises which are caused by the radiofrequency waves will not appear in the obtained ultrasonic image, allowing preventing the degradation of the image quality.

In accordance with the thirteenth aspect described above the information with respect to the radiation of the radiofrequency waves is output to the ultrasonic diagnosis apparatus, and in the ultrasonic diagnosis apparatus the transmission and the reception of the ultrasonic waves by using the ultrasonic probe are performed when the radiofrequency waves are not radiated from the biopsy needle, the noises which are caused by the radiofrequency waves will not appear in the obtained ultrasonic image, allowing preventing the degradation of the image quality.

In accordance with the fourteenth aspect described above, the transmission and the reception of the ultrasonic waves by using the ultrasonic probe are performed when the radiofrequency waves are not radiated from the biopsy needle, so that the noises which are caused by the radiofrequency waves will not appear in the obtained ultrasonic image, allowing preventing the degradation of the image quality.

In accordance with the fifteenth aspect described above, in the ultrasonic diagnosis and treatment system including: an ultrasonic diagnosis apparatus having the ultrasonic probe, the transmission controller unit and the image generator unit; and a radiofrequency wave cautery treatment device having the biopsy needle, the degradation of the image quality of the ultrasonic image which are caused by the radiofrequency waves will be prevented.

Further objects and advantages of the present invention will be apparent from the following description of the embodiments of the invention as illustrated in the accompanying drawings.

### BRIEF DESCRIPTION OF THE DRAWINGS

FIG. 1 is a schematic block diagram illustrating the configuration of the ultrasonic diagnosis and treatment system in accordance with a first embodiment;

FIG. 2 is a schematic block diagram illustrating the configuration of the transmission and reception unit shown in FIG. 1;

FIG. 3 is a schematic block diagram illustrating the configuration of the B-mode processing unit shown in FIG. 1;

FIG. 4 is a schematic block diagram illustrating the configuration of the Doppler processing unit shown in FIG. 1;

FIG. 5 is a schematic block diagram illustrating the configuration of the image processing unit shown in FIG. 1;

FIG. 6 is a diagram illustrating the input and output signals in the AND circuit shown in FIG. 1;

FIG. 7 is a flow chart illustrating an example of the ultrasonic diagnosis and treatment system in accordance with the first embodiment;

FIG. 8 is a schematic block diagram illustrating the configuration of a variation of the ultrasonic diagnosis and treatment system in accordance with the first embodiment;

FIG. 9 is a diagram illustrating the input and output signals in the NOR circuit shown in FIG. 7;

FIG. 10 is a schematic block diagram illustrating the configuration of the ultrasonic diagnosis and treatment apparatus in accordance with a second embodiment; and

FIG. 11 is a schematic block diagram illustrating the configuration of the ultrasonic diagnosis and treatment system in accordance with a third embodiment.

### DETAILED DESCRIPTION OF THE INVENTION

Some embodiments in accordance with the invention will be described in greater details with reference to the accompanying drawings, herein below.

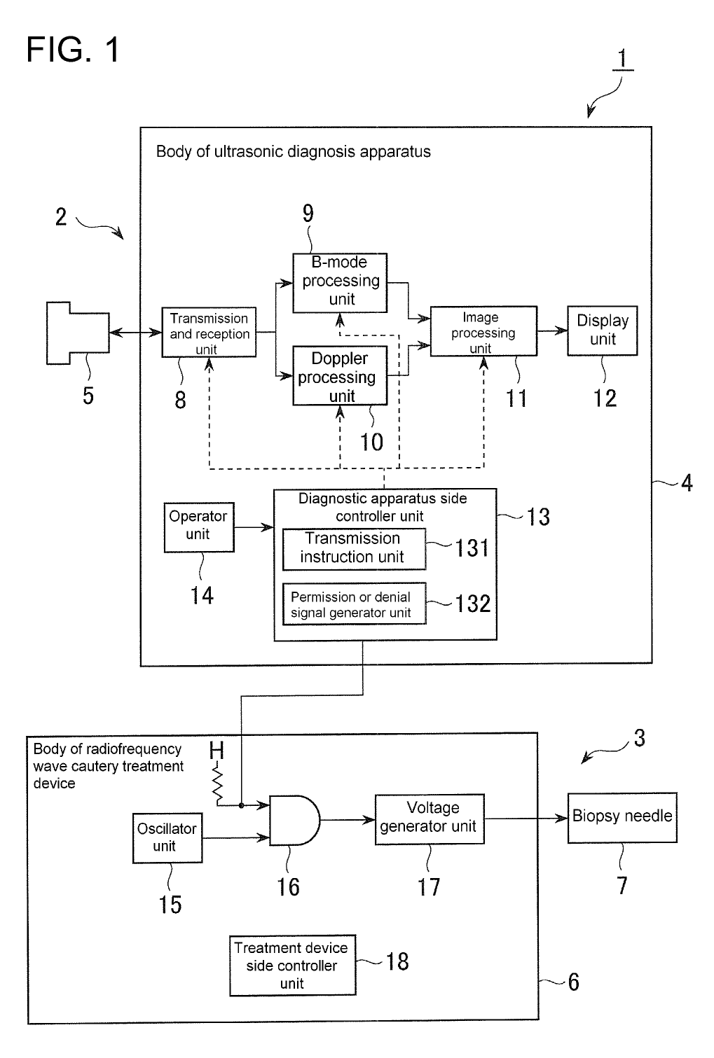

At first, a first embodiment of the invention will be described in greater details. FIG. 1 shows a schematic block diagram illustrating the configuration of the ultrasonic diagnosis and treatment system in accordance with the first embodiment of the invention.

The ultrasonic diagnosis and treatment system 1 as shown in FIG. 1 includes an ultrasonic diagnosis apparatus 2 and a radiofrequency wave cautery treatment device 3. The ultrasonic diagnosis apparatus 2 is comprised of the body of an ultrasonic diagnosis apparatus 4 and an ultrasonic probe 5 connected to the body of the ultrasonic diagnosis apparatus 4. The radiofrequency wave cautery treatment device 3 is comprised of the body of the radiofrequency wave cautery treatment device 6 and a biopsy needle 7 connected to the body of the radiofrequency wave cautery treatment device 6.

The ultrasonic probe 5 has an array of a plurality of ultrasonic wave transducers not shown in the figure. Each of the ultrasonic wave transducers may be made of a piezoelectric material such as the PZT ceramics (lead zirconate titanate).

The biopsy needle 7 is attached via a biopsy adapter (not shown in the figure) to the ultrasonic probe 5.

The body of the ultrasonic diagnosis apparatus 4 includes a transmission and reception unit 8, a B-mode processing unit 9, a Doppler processing unit 10, an image processing unit 11, a display unit 12, a diagnostic apparatus side controller unit 13, and an operator unit 14. Also, the body of the radiofrequency wave cautery treatment device 6 includes an oscillator unit 15, an AND circuit 16, a voltage generator unit 17, and a treatment device side controller unit 18.

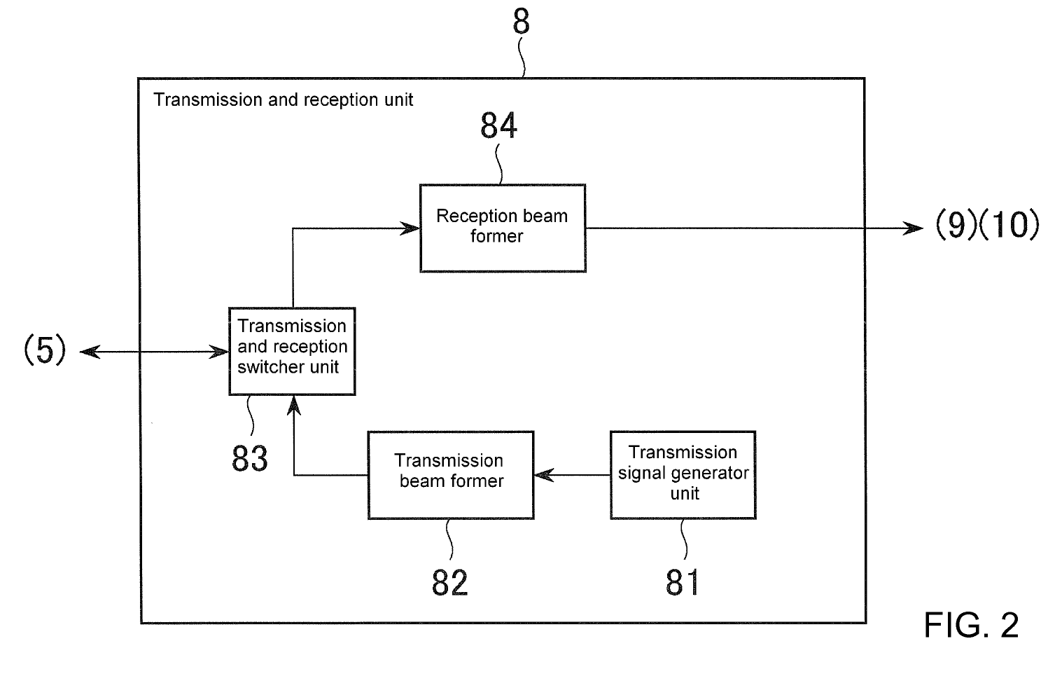

The ultrasonic probe 5 is connected to the transmission and reception unit 8. The transmission and reception unit 8 will be described with reference to FIG. 2. The transmission and reception unit 8 includes a transmission signal generator unit 81, a transmission beam former 82, a transmission and reception switcher unit 83, and a reception beam former 84.

The transmission signal generator unit 81 and the transmission beam former 82 are for providing the driving signal to the ultrasonic probe 5 to transmit ultrasonic waves, which are an example of the best mode for carrying out the transmitter unit of the invention. The transmission signal generator unit 81 and the transmission beam former 82 will be described. The transmission signal generator unit 81 receives the transmission instruction signal from the diagnostic apparatus side controller unit 13, as will be described later in this document, to generate periodically the transmission signals to be input to the transmission beam former 82. The cycle of the transmission signal is controlled by a transmission instruction unit 131 (as will be described later) of the diagnostic apparatus side controller unit 13.

The transmission beam former 82 is for the beam forming of the transmitted waves. The transmission beam former 82 generates the transmission beam forming signal for forming the ultrasonic beam in a predetermined direction based on the transmission signal. The beam forming signal is comprised of a plurality of driving signals which are provided with the difference of time corresponding to the direction. The beam forming is controlled by the diagnostic apparatus side controller unit 13. The transmission beam former 82 outputs the transmission beam forming signal to the transmission and reception switcher unit 83.

The transmission and reception switcher unit 83 outputs the transmission beam forming signal to the ultrasonic transducer array. In the ultrasonic transducer array, a plurality of ultrasonic transducers that form the transmission aperture, each generates the ultrasonic waves having the difference of phase corresponding to the difference of time of the driving signals. By the wave field synthesis of these ultrasonic waves, the ultrasonic beam along the acoustic ray in the predetermined direction is formed.

To the transmission and reception switcher unit 83 the reception beam former 84 is connected. The transmission and reception switcher unit 83 outputs to the reception beam former 84 a plurality of echo signals received by the reception aperture within the ultrasonic wave transducer array.

The reception beam former 84 is for the beam forming of the received waves corresponding to the acoustic ray of the transmitted waves, which adjusts the phases by adding the difference of time to a plurality of received echo, then adds them to generate the echo signal along the acoustic ray in the predetermined direction. The beam forming of the received waves is controlled by the diagnostic apparatus side controller unit 13.

The transmission and reception unit 8 is connected to the B-mode processing unit 9 and to the Doppler processing unit 10. The echo signals for each acoustic ray, which is output from the transmission and reception unit 8, are input into the B-mode processing unit 9 and into the Doppler processing unit 10.

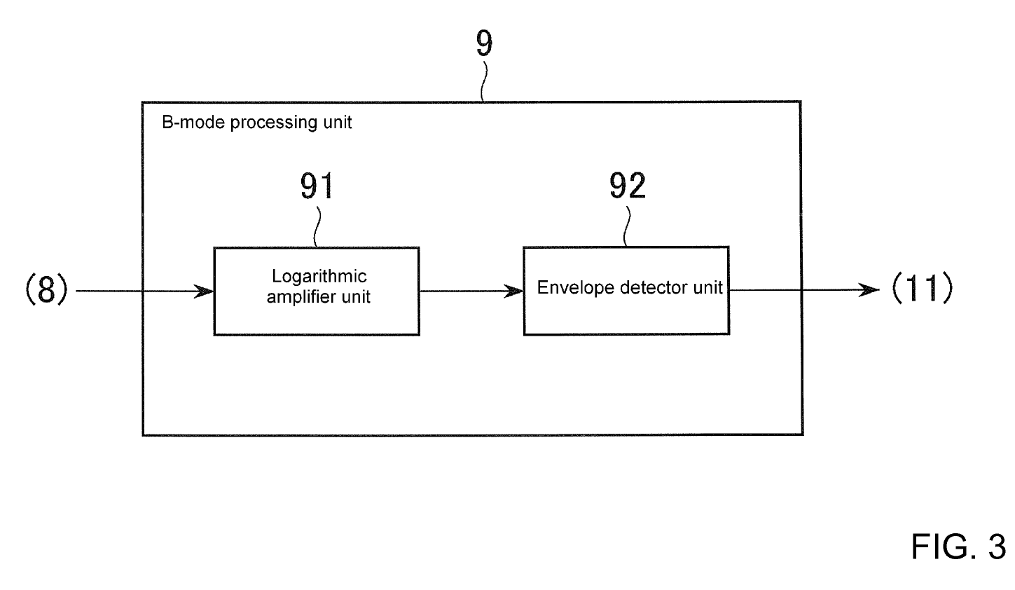

The B-mode processing unit 9 generates B-mode image data for each acoustic ray based on the echo signals. FIG. 3 shows a schematic block diagram illustrating the configuration of the B-mode processing unit 9. The B-mode processing unit 9 as shown in FIG. 3 includes a logarithmic amplifier unit 91 and an envelope detector unit 92.

The B-mode processing unit 9 performs the logarithmic amplification of the echo signals by the logarithmic amplifier unit 91, and the envelope detection by the envelope detector unit 92 to obtain the signal indicative of the intensity of the echo at the reflection point of each of the acoustic ray, namely A scope signals, then the amplitude of each instance on the A scope signal is used as the brightness value in order to generate B-mode image data.

The Doppler processing unit 10 is for generating Doppler image data for each acoustic ray, based on the echo signals. The Doppler image data includes the flow velocity data, the variance data, and the power data, as will be described in greater details later in this document.

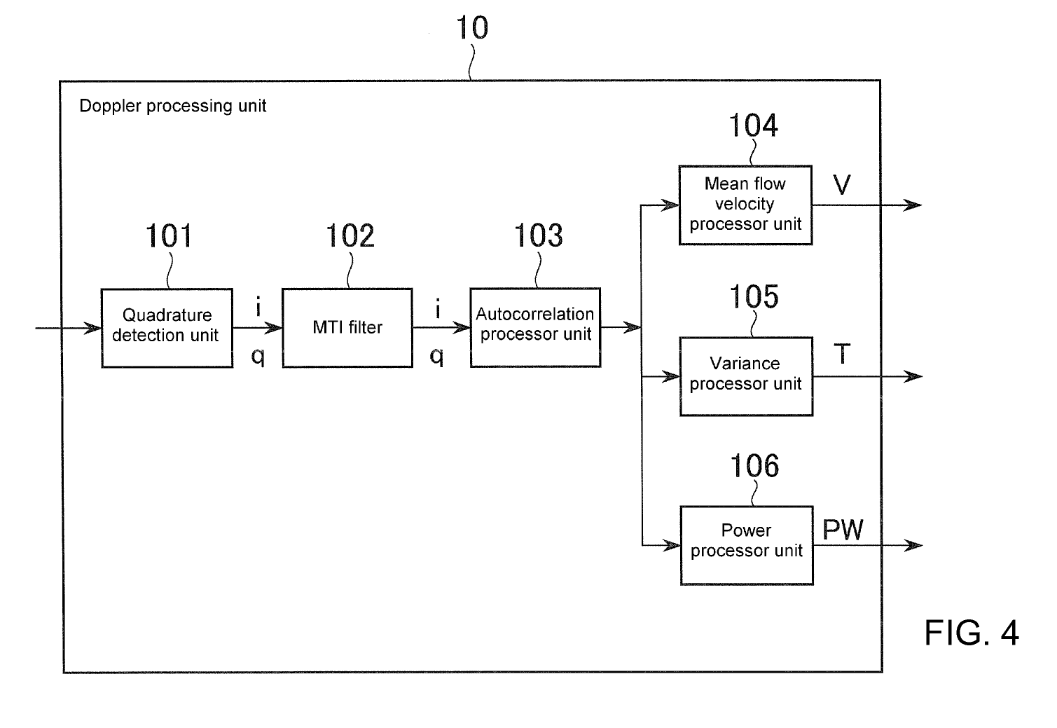

Now referring to FIG. 4 there is shown a schematic block diagram illustrating an example of the configuration of the Doppler processing unit 10. As shown in FIG. 4, the Doppler processing unit 10 includes a quadrature detection unit 101, an MTI (moving target indication) filter 102, an autocorrelation processor unit 103, a mean flow velocity processor unit 104, a variance processor unit 105, and a power processor unit 106.

The Doppler processing unit 10 performs the quadrature detection of the echo signals in the quadrature detection unit 101, then MTI processing in the MTI filter 102 to determine the amount of Doppler shift of the echo signals. Also it performs in the autocorrelation processor unit 103 the autocorrelation processing with respect to the output signal from the MTI filter 102. Then the mean flow velocity processor unit 104 determines the mean flow velocity V from the result of the autocorrelation processing, the variance processor unit 105 determines the variance T of the flow from the result of the autocorrelation processing, then the power processor unit 106 determines the power PW of the Doppler signal from the result of the autocorrelation processing. The mean flow velocity will be referred to as simply the flow velocity herein below. The variance of the flow velocity will also be referred to as simply the variance, and the power of the Doppler signal will be referred to as simply the power, herein below.

The Doppler processing unit 10 will obtain the data indicative of respectively the flow velocity V, the variance T, and the power PW of the echo source that is moving within the subject for each acoustic ray. These items of data indicate the flow velocity, the variance, and the power of the pixel on the acoustic ray. It should be noted here that the flow velocity may be obtained as the components in the direction of the acoustic ray. Also it should be noted that the direction reaching to the ultrasonic probe 5 and the direction separated away therefrom are distinguished.

The B-mode processing unit 9 and the Doppler processing unit 10 are connected to the image processing unit 11. The image processing unit 11 generates a B-mode image and a Doppler image based on the data input from the B-mode processing unit 9 and from the Doppler processing unit 10, respectively. The B-mode processing unit 9, the Doppler processing unit 10, and the image processing unit 11 are an example of the preferred embodiment of the image generator unit in accordance with the invention.

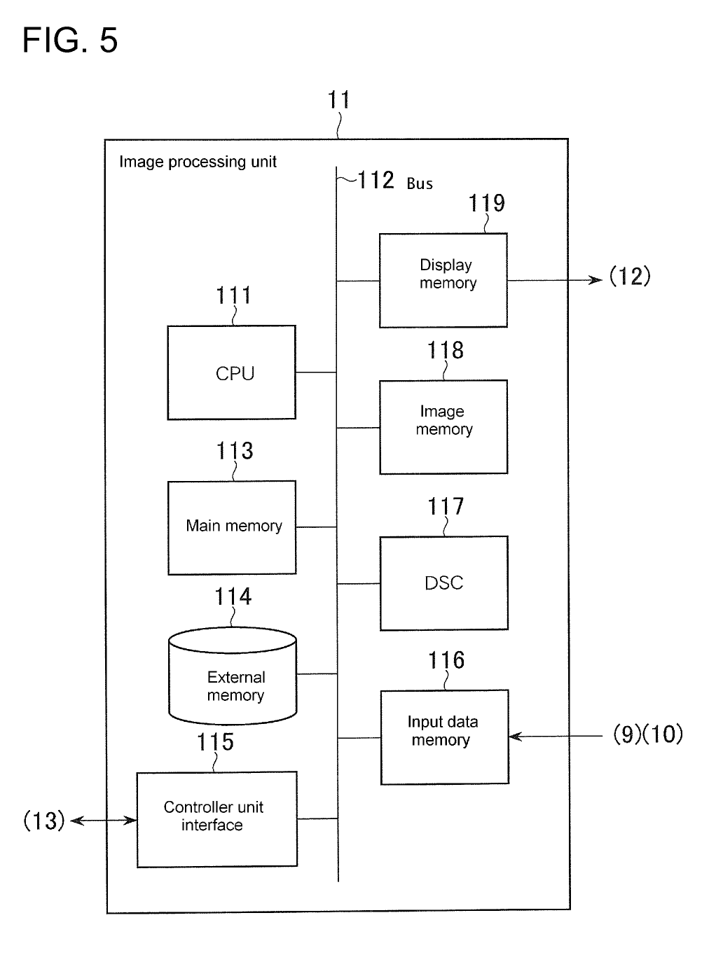

The image processing unit 11 will be described in greater details with reference to FIG. 5. FIG. 5 shows a schematic block diagram illustrating an example of the configuration of the image processing unit 11. As shown in FIG. 5, the image processing unit 11 includes a CPU (central processing unit) 111. The CPU 111 is connected through a bus 112 to a main memory 113, an external memory 114, a controller unit interface 115, an input data memory 116, a digital scan converter (DSC) 117, an image memory 118, and a display memory 119.

The external memory 114 stores a program to be executed by the CPU 111. The external memory 114 also stores a variety of data used when the CPU 111 executes the program.

The CPU 111 loads and executes the program from the external memory 114 to the main memory 113 to perform a predetermined image processing. The CPU 111 transmits and receives control signals to and from the diagnostic apparatus side controller unit 13 through the controller unit interface 115 during the program execution.

The B-mode image data and the Doppler image data, which are input for each acoustic ray from the B-mode processing unit 9 and from the Doppler processing unit 10 respectively, are stored respectively in the input data memory 116. The data in the input data memory 116 will be scan converted by the digital scan converter 117 and stored in the image memory 118. The data in the image memory 118 will be output to the display unit 12 through the display memory 119.

The image processing unit 11 is connected to the display unit 12. The display unit 12 is provided with image signals from the image processing unit 11 in order to display an image based thereof. The display unit 12 may be comprised of a CRT or a liquid crystal display that is capable of displaying color images.

The transmission and reception unit 8, the B-mode processing unit 9, the Doppler processing unit 10, the image processing unit 11, and the display unit 12 as have been described above are connected to the diagnostic apparatus side controller unit 13, and a variety of signals is input from the diagnostic apparatus side controller unit 13. Then under the control of the diagnostic apparatus side controller unit 13 the B-mode operation and the Doppler mode operation will be executed.

The diagnostic apparatus side controller unit 13 includes the transmission instruction unit 131, and a generator unit 132 of the signal permitting or denying the radiofrequency wave radiation from the biopsy needle 7. The transmission instruction unit 131 outputs the transmission instruction signal of the ultrasonic waves to control the generation and the output of the transmission signal by the transmission signal generator unit 81. The permission or denial signal generator unit 132 generates a radiofrequency wave radiation permission signal for permitting the radiation of the radiofrequency waves from the biopsy needle 7 and a radiofrequency wave radiation denial signal for denying the radiation of the radiofrequency waves from the biopsy needle 7, to output to the AND circuit 16 of the body of the radiofrequency wave cautery treatment device 6.

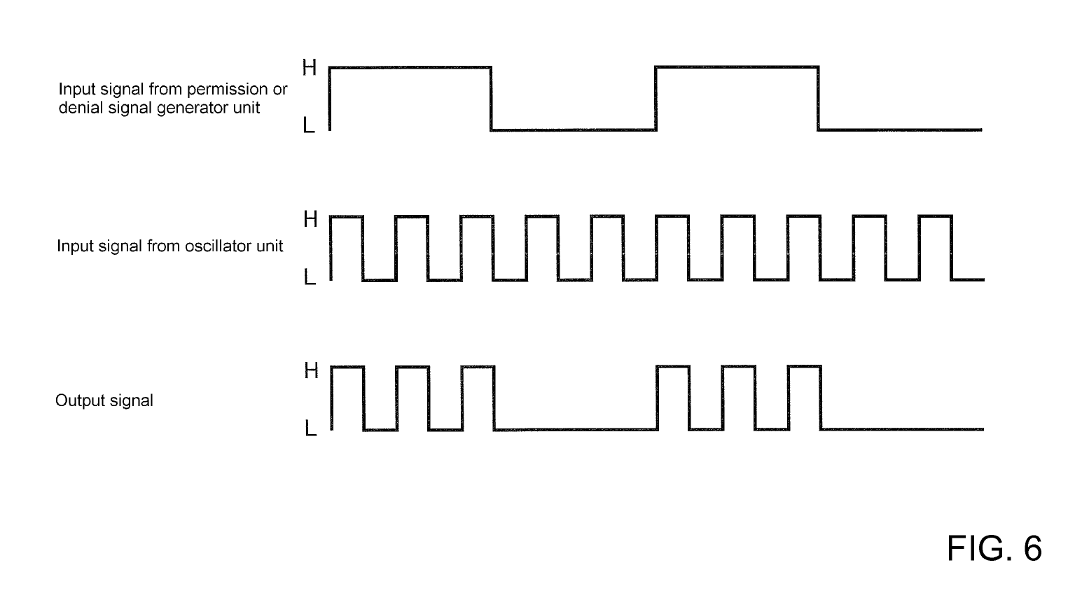

Now referring to FIG. 6 there is shown an example of the permission or denial signal to be output from the permission or denial signal generator unit 132 and to be input to the AND circuit 16. The permission or denial signal may be either the radiofrequency wave radiation permission signal in H level, or the radiofrequency wave radiation denial signal in L level. The radiofrequency wave radiation permission signal is the signal to be output when the radiofrequency waves are to be radiated from the biopsy needle 7. The permission or denial signal generator unit 132 which generates and outputs such a radiofrequency wave radiation permission signal is an example of the preferred embodiment of the permission signal generator unit in accordance with the invention.

On the other hand, the radiofrequency wave radiation denial signal is output when the transmission instruction signal is output from the transmission instruction unit 131 to transmit and receive the ultrasonic waves. The radiofrequency wave radiation permission signal and the radiofrequency wave radiation denial signal are alternately output, and as will be described in greater details later in this document, the ultrasonic waves is transmitted and received to generate an ultrasonic image only when the radiofrequency wave radiation denial signal is output to stop radiating the radiofrequency waves from the biopsy needle 104. The lengths of the duration of output time of the radiofrequency wave radiation permission signal and the radiofrequency wave radiation denial signal may be the period of time of the transmission and the reception of the ultrasonic waves for each acoustic ray, or the period of time of the transmission and the reception of the ultrasonic waves for one frame or a plurality of frames.

When the radiofrequency wave radiation denial signal is output from the permission or denial signal generator unit 132, the ultrasonic waves is transmitted based on the transmission instruction signal from the transmission instruction unit 131 while the echo signals are received. On the other hand, when the radiofrequency wave radiation permission signal is output from the permission or denial signal generator unit 132, as will be described in greater details later, the radiofrequency wave radiation permission signal is radiated from the biopsy needle 7. More specifically, the diagnostic apparatus side controller unit 13 controls to alternately perform the output of the transmission signal from the transmission instruction unit 131 and the output of the radiofrequency wave radiation permission signal from the permission or denial signal generator unit 132, as well as to alternately perform the transmission and the reception of the ultrasonic waves by using the ultrasonic probe 5 and the radiation of the radiofrequency waves by the biopsy needle 7. This is an example of an embodiment of the controller unit in accordance with the invention.

Now the relationship between the length of the period of time of the radiation of the radiofrequency wave, that is, the length of the output time of the radiofrequency wave radiation permission signal, and the length of the period of time of the transmission and the reception of the ultrasonic waves, that is, the length of the output time of the transmission instruction signal and the length of the output time of the radiofrequency wave radiation denial signal will be described. When giving preference to the cautery treatment even though the frame rate of the ultrasonic image decreases, the radiofrequency wave radiation permission signal, the transmission instruction signal, and the radiofrequency wave radiation denial signal will be output such that the radiation time of the radiofrequency wave is longer than the transmission and reception time of the ultrasonic waves. On the other hand, when giving preference to the real time property of the ultrasonic image, the radiofrequency wave radiation permission signal, the transmission instruction signal, and the radiofrequency wave radiation denial signal will be output such that the transmission and reception time of the ultrasonic waves is longer than the radiation time of the radiofrequency wave.

To the diagnostic apparatus side controller unit 13, the operator unit 14 is connected. The operator unit 14 is operated by an operator, so as to input any given instruction and information to the diagnostic apparatus side controller unit 13. The operator unit 14 includes for example a keyboard or a pointing device as well as any other operating device.

Next, the configuration of the body of the radiofrequency wave cautery treatment device 6 will be described in greater details. From the oscillator unit 15 of the body of the radiofrequency wave cautery treatment device 6, the radiofrequency wave radiation instruction pulses are output for instructing the radiofrequency wave to be radiated from the biopsy needle 7. The radiofrequency wave radiation instruction pulses from the oscillator unit 15 may be output when the control signal from the treatment device side controller unit 18 is input to the oscillator unit 15, by turning on the cautery switch (not shown in the figure) provided on the body of the radiofrequency wave cautery treatment device 6. The oscillator unit 15 is an example of the preferred embodiment of the radiofrequency wave radiation instruction signal generator unit in accordance with the invention.

To the AND circuit 16, the radiofrequency wave radiation instruction pulses from the oscillator unit 15 and the permission or denial signals from the permission or denial signal generator unit 132 are input. The AND circuit 16 will make an AND of the radiofrequency wave radiation instruction pulses with the permission or denial signal to output to the voltage generator unit 17. Now referring to FIG. 6 there is shown signals to be output from the AND circuit 16 to the voltage generator unit 17. As shown in FIG. 6, the AND circuit 16 will output to the voltage generator unit 17 the radiofrequency wave radiation instruction pulses from the oscillator unit 15 if the radiofrequency wave radiation permission signal is input. The voltage generator unit 17 applies a voltage to the biopsy needle 7 to radiate the radiofrequency wave when the input of the radiofrequency wave radiation instruction pulses is present. Therefore the AND circuit 16 outputs the radiofrequency wave radiation instruction pulses to radiate the radiofrequency wave from the biopsy needle 7 only when the radiofrequency wave radiation instruction pulse is input and if the radiofrequency wave radiation permission signal is input, which is an example of the preferred embodiment of the radiofrequency wave radiation permission unit in accordance with the invention.

On the other hand, the AND circuit 16 will not output any radiofrequency wave radiation instruction pulses if the radiofrequency wave radiation denial signal is input even through the radiofrequency wave radiation instruction pulse is input, so that the radiation of the radiofrequency wave from the biopsy needle 7 will not be performed.

By the way, the body of the radiofrequency wave cautery treatment device 6 has an H level signal generator unit H. When the radiofrequency wave cautery treatment is conducted without using the ultrasonic diagnosis apparatus 2 but only with the radiofrequency wave cautery treatment device 3, because the permission or denial signal from the permission or denial signal generator unit 132 is not applied to the AND circuit 16, the signals from the H level signal generator unit H is to be input instead. By doing this the radiofrequency wave radiation instruction pulses from the AND circuit 16 will be output to allow radiating the radiofrequency wave from the biopsy needle 7, enabling the radiofrequency wave cautery treatment with the radiofrequency wave cautery treatment device 3 alone.

The treatment device side controller unit 18 controls the radiation of the radiofrequency wave from the biopsy needle 7 as well as controls components of the body of the radiofrequency wave cautery treatment device 6.

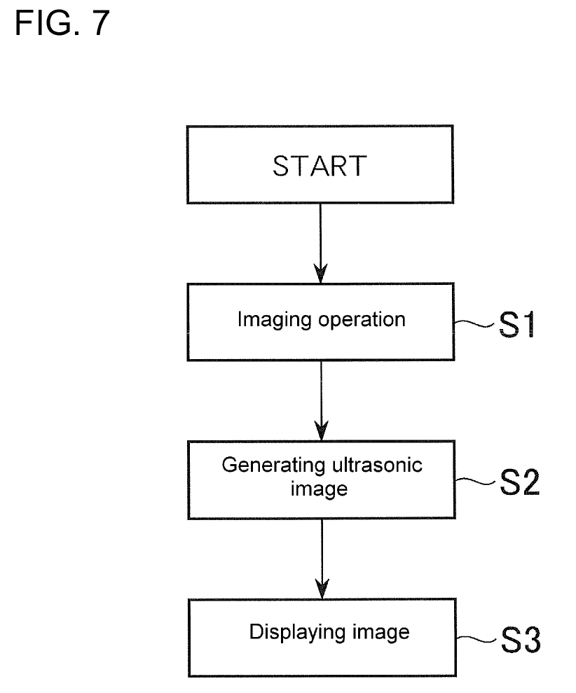

Now the operation of the ultrasonic diagnosis and treatment system 1 will be described in greater details with reference to FIG. 7. In the ultrasonic diagnosis and treatment system 1, in order to insert the biopsy needle to the lesion while viewing the ultrasonic image to conduct the cautery, at first in step S1, the echo signals are received by the ultrasonic probe 5 by doing the imaging operation, then in step S2, based on the obtained echo signals the ultrasonic image will be generated. Then in step S3 the ultrasonic image will be displayed.

Those steps mentioned above will be described in greater details. First, the step S1 is described, in step S1, the ultrasonic probe 5 is placed in contact with the subject, and the operator unit 14 is operated to perform the imaging operation for example using the B-mode and the Doppler mode. By doing this, under the control of the diagnostic apparatus side controller unit 13, the B-mode imaging and the Doppler mode imaging are performed as in the time sharing basis. More specifically, for example, by performing one B-mode scan each time a predetermined number of scans in the Doppler mode is executed, a mix scans of the B-mode and the Doppler mode may be conducted.

In the B-mode the transmission and reception unit 8 scans the inside of the subject in the sequential order of the acoustic rays through the ultrasonic probe 5 to receive sequentially the echo thereof. The transmission of the ultrasonic waves from the ultrasonic probe 5 is performed in accordance with the transmission signal generated in the transmission signal generator unit 81. Also in the Doppler mode, the transmission and reception unit 8 scans the inside of the subject in the sequential order of the acoustic rays through the ultrasonic probe 5 to receive the echo thereof. At that time a plurality of numbers of the transmission of ultrasonic waves and of the reception of the echo is performed for each single acoustic ray.

Next, the step S2 will be described, the B-mode processing unit 9 will form the B-mode image data for each acoustic ray based on the echo signals to be input from the transmission and reception unit 8. The image processing unit 11 will store into the input data memory 116 the B-mode image data for each single acoustic ray to be input from the B-mode processing unit 9. By doing this an acoustic ray data space with respect to the B-mode image data is formed in the input data memory 116.

In addition, the Doppler processing unit 10 determines the flow velocity V, the variance T, and the power PW based on the echo signals. These calculation values will become the data indicating the variance and the power of the echo source, for each pixel and for each acoustic ray the velocity.

The image processing unit 11 will store in the input data memory 116 each Doppler image data for each acoustic ray and for each pixel, which is to be input from the Doppler processing unit 10. By doing this an acoustic ray data space with respect to each Doppler image data is formed within the input data memory 116.

The CPU 111 will scan convert the B-mode image data and each Doppler image data in the input data memory 116 with the digital scan converter 117 to write down data into the image memory 118. At this time the Doppler image data will be written thereto as the flow velocity distribution image data by combining the flow velocity V and the variance T, the power Doppler image data by using the power PW, or the power Doppler image data with the variance by combining the power PW and the variance T, and the variance image data by using the variance T.

The CPU 111 will write the B-mode image data and each Doppler image data to a different area of the image memory 118. Then the CPU 111 in the part of the processing in the step S3, displays the image based on these B-mode image data and each Doppler image data on the display unit 12.

The B-mode image indicates a tomographic image of the tissue inside the body at the scan plane of the acoustic ray. Among the color Doppler images, the flow velocity distribution image is the image indicative of the two dimensional distribution of the flow velocity of the echo source. In this image the display color differs in correspondence with the direction of the flow, and the brightness of the display color differs in correspondence with the flow velocity, as well as the purity of the display color varies by increasing the amount of color mixture of a predetermined color in correspondence with the variance.

The power Doppler image of the color Doppler images is the image indicative of the two dimensional distribution of the power of the Doppler signals. This image indicates the location of the moving echo source. Then the brightness of the display color of the image corresponds to the power. When this is combined with the variance, the purity of the display color varies by increasing the amount of the color mixture of a predetermined color in correspondence with the variance. The variance image is the image indicative of the two dimensional image of the variance values. This image also indicates the location of the moving echo source. The brightness of the display color corresponds to the amount of the variance.

When displaying the image as described above onto the display unit 12, the image will be synthesized with the B-mode image in the display memory 119, then the synthetic image will be displayed on the display unit 12 so that a color Doppler image may be presented in which the positional relationship of the tissues within the body is clearly depicted.

On the display unit 12, the ultrasonic images are displayed in real time basis by iteratively repeating the steps S1 to S3 as described above. The operator inserts the biopsy needle 7 into the subject while viewing the real time ultrasonic image that is displayed on the display unit 12, and operates the operator unit 14 to radiate the radiofrequency waves from the biopsy needle 7. In the imaging operation in step S1, the radiation of the radiofrequency waves from the biopsy needle 7 and the transmission and the reception of the ultrasonic waves are alternately performed. More specifically, for example if the output time of the radiofrequency wave radiation denial signal is equal to one acoustic ray of the transmission and the reception time of the ultrasonic waves, the transmission and the reception of the ultrasonic waves for one acoustic ray is performed, thereafter the radiofrequency waves are radiated, then the radiation of the radiofrequency waves is stopped and again the transmission and the reception of the ultrasonic waves for one acoustic ray is performed, and so on, this is iteratively repeated. Even if the output time of the radiofrequency wave radiation denial signal is for a plurality of acoustic rays, for one single frame, or for a plurality of frames, the radiation of the radiofrequency waves and the transmission and the reception of the ultrasonic waves will be alternately performed.

As can be appreciated from the foregoing description, in accordance with the ultrasonic diagnosis and treatment system 1, the radiation of the radiofrequency waves and the transmission and the reception of the ultrasonic waves are alternately performed, and since when the transmission and the reception of the ultrasonic waves is performed the radiation of the radiofrequency waves is not performed, the noises which are caused by the radiofrequency waves will not appear in the obtained ultrasonic image, allowing preventing the degradation of the image quality.

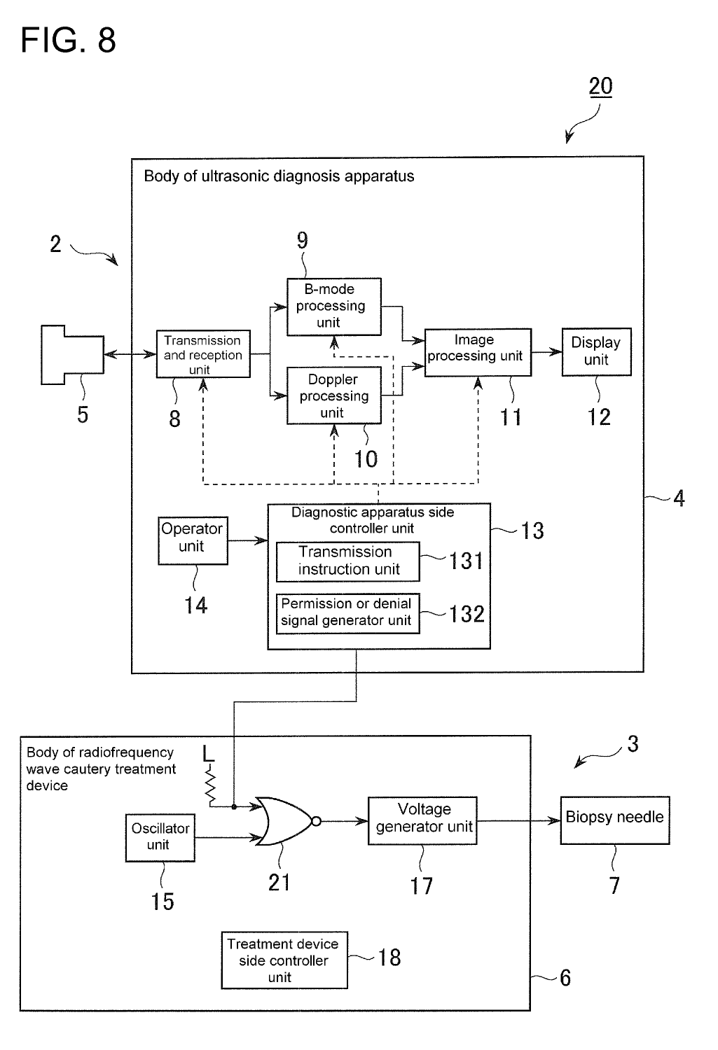

Next, a variation of the first preferred embodiment will be described in greater details now. Now referring to FIG. 8 there is shown a schematic block diagram illustrating the configuration of the ultrasonic diagnosis and treatment system in accordance with the variation of the first preferred embodiment of the invention. The body of the radiofrequency wave cautery treatment device 6 in the ultrasonic diagnosis and treatment apparatus 20 as shown in FIG. 8 includes a NOR circuit 21, instead of the AND circuit 16. The NOR circuit 21 is another example of an embodiment of the radiofrequency wave radiation permission unit in accordance with the invention.

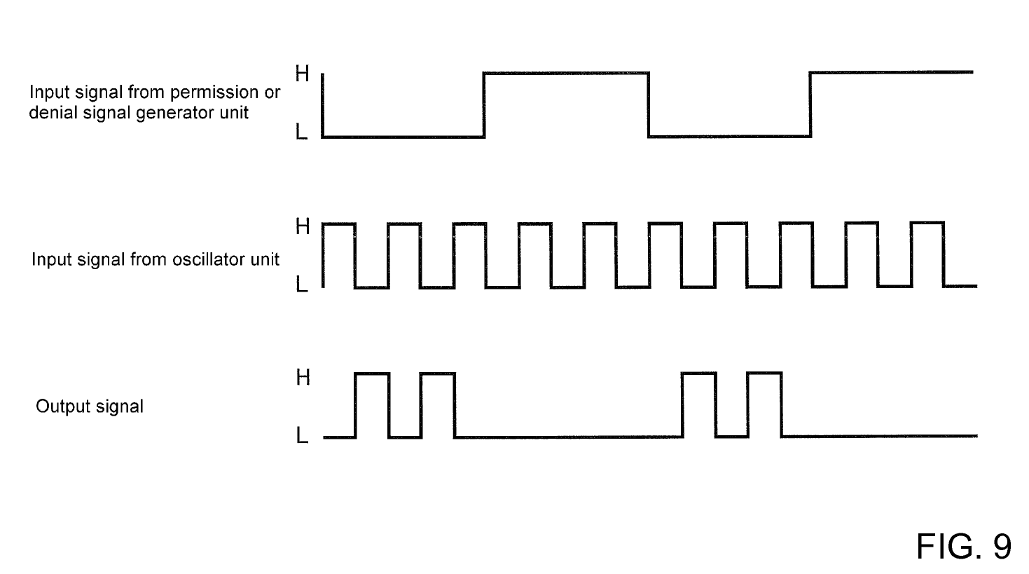

In this variation the permission or denial signal to be output from the permission or denial signal generator unit 132 and to be input to the NOR circuit 21 has an L level indicative of the radiofrequency wave radiation permission signal, and an H level indicative of the radiofrequency wave radiation denial signal. FIG. 9 shows the permission or denial signal of this variation. The NOR circuit 21 will output to the voltage generator unit 17 the inversion of the radiofrequency wave radiation instruction pulses from the oscillator unit 15, as is shown in FIG. 9, when the L level of the radiofrequency wave radiation permission signal is input. By doing this a voltage is applied from the voltage generator unit 17 to the biopsy needle 7 to radiate the radiofrequency waves.

By the way in the body of the radiofrequency wave cautery treatment device 6 of this variation, there is an L level signal generator unit L equipped instead of the H level signal generator unit H, and when the radiofrequency wave cautery treatment is conducted without using the ultrasonic diagnosis apparatus 2, the signal from the L level signal generator unit L is to be input to the NOR circuit 21 instead of the permission or denial signal from the permission or denial signal generator unit 132.

The radiation of the radiofrequency waves and the transmission of the ultrasonic waves are alternately performed in the ultrasonic diagnosis and treatment apparatus 20 of the variation as have been described above, and because the radiofrequency waves are not radiated when the ultrasonic waves are transmitted and received, so that the noises which are caused by the radiofrequency waves will not appear in the obtained ultrasonic image, allowing preventing the degradation of the image quality.

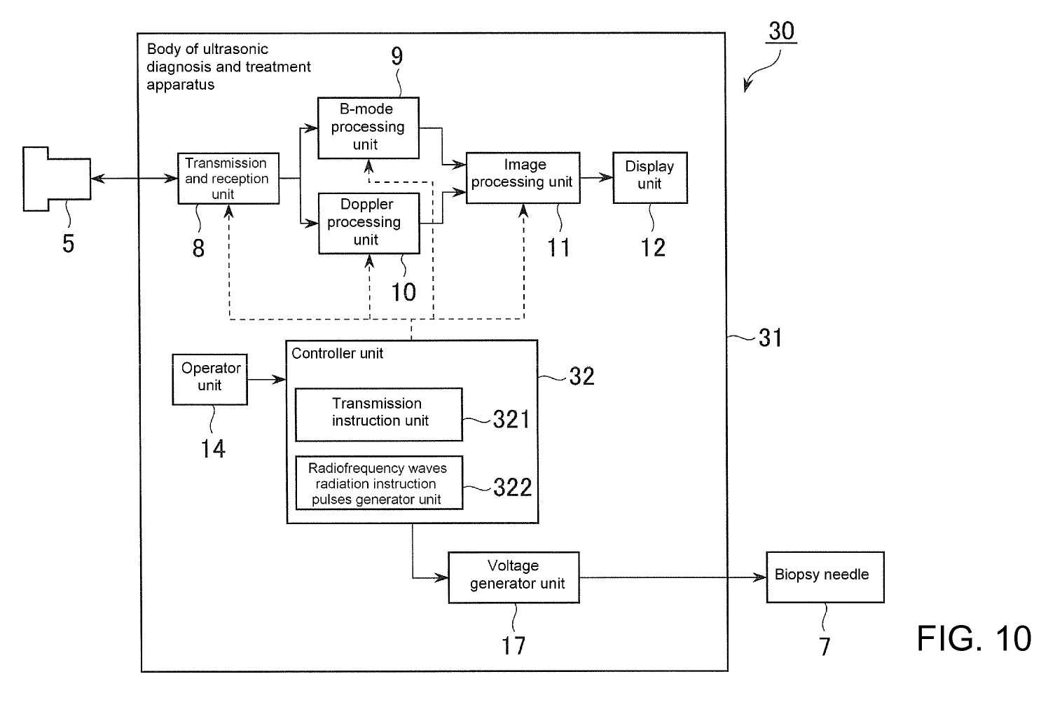

Now a second embodiment of the invention will be described in greater details herein below with reference to FIG. 10. FIG. 10 shows a schematic block diagram illustrating the configuration of the ultrasonic diagnosis and treatment apparatus in accordance with the second embodiment of the invention. In the following description, only the configuration that is different from the first embodiment will be discussed. With respect to the operation, the operation is almost fundamentally similar to the steps S1 to S3 of FIG. 7 that have been described in the above first embodiment, and only the operation that is differed therefrom will be described.

In this second embodiment, as shown in FIG. 10, the body of the ultrasonic diagnosis apparatus and the body of the radiofrequency wave cautery treatment device are integrated into one body to form the ultrasonic diagnosis and treatment system 1 as an ultrasonic diagnosis and treatment apparatus 30. More specifically, the ultrasonic diagnosis and treatment apparatus 30 includes: the body of the apparatus 31 having the transmission and reception unit 8, the B-mode processing unit 9, the Doppler processing unit 10, the image processing unit 11, the display unit 12, the operator unit 14, the controller unit 32, and the voltage generator unit 17; and the ultrasonic probe 5 and the biopsy needle 7 connected to the body of the apparatus 31. The controller unit 32 performs the control similar to the diagnostic apparatus side controller unit 13 and the treatment device side controller unit 18 as shown in FIG. 1. The ultrasonic diagnosis and treatment apparatus 30 is formed without the oscillator unit 15, the AND circuit 16 or the NOR circuit 21, and radiates the radiofrequency waves based on the radiofrequency waves radiation instruction pulses output from the radiofrequency waves radiation instruction pulses generator unit 322 (will be described later).

The controller unit 32 has a transmission instruction unit 321 and the radiofrequency waves radiation instruction pulses generator unit 322. The transmission instruction unit 321, similar to the transmission instruction unit 131 of the diagnostic apparatus side controller unit 13 in the first embodiment, outputs the transmission instruction signal of the ultrasonic waves to control the generation and the output of the transmission signal by the transmission signal generator unit 81. The radiofrequency waves radiation instruction pulses generator unit 322 outputs to the voltage generator unit 17 the radiofrequency waves radiation instruction pulses served as the radiofrequency wave radiation instruction signal for radiating the radiofrequency waves from the biopsy needle 7. The radiofrequency waves radiation instruction pulses generator unit 322 is an example of the embodiment of the radiofrequency wave radiation instruction signal generator unit in accordance with the invention. The voltage generator unit 17 applies a voltage to the biopsy needle 7 to radiate the radiofrequency waves when the radiofrequency wave radiation instruction pulses is input.

In the imaging operation, the output of the transmission instruction signal from the transmission instruction unit 321 and the output of the radiofrequency wave radiation instruction pulses from the radiofrequency waves radiation instruction pulses generator unit 322 are alternately performed. Therefore, as the radiation of the radiofrequency waves and the transmission and the reception of the ultrasonic waves are alternately performed, no radiofrequency wave is radiated when the transmission and reception of the ultrasonic waves is in progress, so that the noises which are caused by the radiofrequency waves will not appear in the obtained ultrasonic image, allowing preventing the degradation of the image quality.

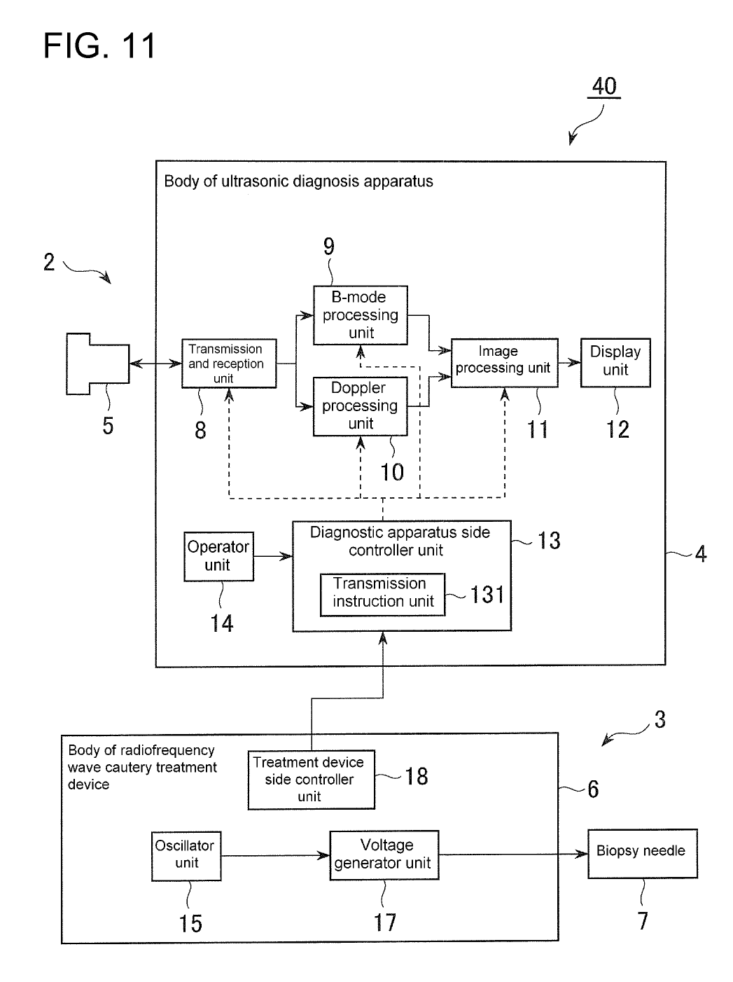

Next a third embodiment of the invention will be described in greater details with reference to FIG. 11. FIG. 11 shows a schematic block diagram illustrating the configuration of the ultrasonic diagnosis and treatment system in accordance with the third embodiment of the invention.

The ultrasonic diagnosis and treatment system 40 in accordance with the third embodiment of the invention includes, as similar to the first embodiment, the ultrasonic diagnosis apparatus 2 and the radiofrequency wave cautery treatment device 3. In the description that follows, only the configuration that is different from the first embodiment will be described. The operation is almost fundamentally similar to the steps S1 to S3 shown in FIG. 7 and described in the first embodiment, and only the part that is different therefrom will be described herein below.

The body of the radiofrequency wave cautery treatment device 6 does not have a AND circuit 16, so that the radiofrequency wave radiation instruction pulses from the oscillator unit 15 will be input to the voltage generator unit 17 directly. By doing this in the radiofrequency wave cautery treatment device 3 in this embodiment, when turning on the cautery switch, the radiofrequency waves are always radiated from the biopsy needle 7.

In addition, the treatment device side controller unit 18 outputs to the diagnostic apparatus side controller unit 13 the radiofrequency wave radiation signal indicating that the radiofrequency wave radiation instruction pulses has been input from the oscillator unit 15 to the voltage generator unit 17 and the radiofrequency waves is being radiated from the biopsy needle 7. The radiofrequency wave radiation signal is an example of the preferred embodiment of the information with respect to the radiofrequency wave radiation in accordance with the invention, and the treatment device side controller unit 18 is an example of the preferred embodiment of the radiation controller unit in accordance with the invention.

In this embodiment, the diagnostic apparatus side controller unit 13 in the body of the ultrasonic diagnosis apparatus 4 does not have a denial signal generator unit 132. And the diagnostic apparatus side controller unit 13 controls the output of the transmission instruction signal from the transmission instruction unit 131 based on the presence or absence of the radiofrequency wave radiation signal to be output from the oscillator unit 15. The transmission instruction unit 131 in the present embodiment is an example of the preferred embodiment of the transmission controller unit in accordance with the invention.

More specifically, the diagnostic apparatus side controller unit 13 determines in the imaging operation the presence or absence of the input of the radiofrequency wave radiation signal, and then outputs the transmission instruction signal from the transmission instruction unit 131 when the input of the radiofrequency wave radiation signal is absent. On the other hand, the diagnostic apparatus side controller unit 13 will not output the transmission instruction signal from the transmission instruction unit 131 if the input of the radiofrequency wave radiation signal is present. By doing this the transmission and the reception of the ultrasonic waves by using the ultrasonic probe 5 will be performed while no radiation of the radiofrequency waves is performed, so that the noises that are caused by the radiofrequency waves will not appear in the obtained ultrasonic image, allowing preventing the degradation of the image quality.

As can be appreciated from the foregoing description, although the invention has been described by way of examples of the preferred embodiments as described above, the invention is not limited thereto, and may be practiced with a number of changes and modifications without departing from the spirits and the scope of the invention. For example, although not specifically shown, by integrating the body of the ultrasonic diagnosis apparatus 4 having the configuration as described above in the first preferred embodiment and the body of the radiofrequency wave cautery treatment device 6, without modification, the body of the apparatus 31 as described above in the second preferred embodiment may be formed. Also not specifically shown in the figure, the body of the ultrasonic diagnosis apparatus 4 having the configuration as described above in the third preferred embodiment may be integrated into one body with the body of the radiofrequency wave cautery treatment device 6 to form the body of the apparatus 31 in the second preferred embodiment.

Furthermore, in the third preferred embodiment, the signal indicating that the radiofrequency waves are not radiated may be input from the treatment device side controller unit 18 to the diagnostic apparatus side controller unit 13 as the information with respect to the radiation of the radiofrequency wave, and when this signal is input the transmission instruction signal may be output from the transmission instruction unit 131.

Many widely different embodiments of the invention may be configured without departing from the spirit and the scope of the present invention. It should be understood that the present invention is not limited to the specific embodiments described in the specification, except as defined in the appended claim.

1. An ultrasonic diagnosis apparatus comprising:
an ultrasonic probe configured to transmit ultrasonic waves and receive echo signals;
a radiofrequency wave cautery treatment device configured to radiate radiofrequency waves;
an image generator unit configured to generate an ultrasonic image based on the echo signals received by said ultrasonic probe; and
a controller unit configured to alternately instruct said ultrasonic probe to transmit ultrasonic waves and said radiofrequency wave cautery treatment device to radiate radiofrequency waves.

2. An ultrasonic diagnosis apparatus according to claim 1, wherein:
said controller unit comprises:
a transmission instructor unit configured to output a transmission instruction signal to a transmitter unit configured to provide to said ultrasonic probe a driving signal to transmit ultrasonic waves; and
a permission signal generator unit configured to generate a radiofrequency wave radiation permission signal that enables a biopsy needle within said radiofrequency wave cautery treatment device to radiate radiofrequency waves;
said permission signal generator unit is configured to output the radiofrequency wave radiation permission signal to a radiofrequency wave radiation permission unit of said radiofrequency wave cautery treatment device, said radiofrequency wave cautery treatment device further comprising:
a generator unit configured to generate a radiofrequency wave radiation instruction signal for radiating the radiofrequency waves from said biopsy needle; and
said radiofrequency wave radiation permission unit configured to enable said biopsy needle to radiate the radiofrequency waves by outputting the radiofrequency wave radiation instruction signal only when the radiofrequency wave radiation instruction signal is input from said radiofrequency wave radiation instruction signal generator unit and if the radiofrequency wave radiation permission signal is input from said permission signal generator unit; and
the output of the transmission instruction signal from said transmission instruction unit and the output of the radiofrequency wave radiation permission signal from said permission signal generator unit are alternately performed.

3. An ultrasonic diagnosis apparatus according to claim 1, wherein:
the radiation of the radiofrequency waves from a biopsy needle within said radiofrequency wave cautery treatment device is controlled by said controller unit.

4. An ultrasonic diagnosis apparatus according to claim 3, further comprising:
a generator unit configured to generate radiofrequency wave radiation instruction signal for radiating the radiofrequency waves from said biopsy needle; and
a radiofrequency wave radiation permission unit configured to output the radiofrequency wave radiation instruction signal to radiate the radiofrequency waves from said biopsy needle only when the radiofrequency wave radiation instruction signal is input from said radiofrequency wave radiation instruction signal generator unit and if the radiofrequency wave radiation permission signal is input from said permission signal generator unit.

5. An ultrasonic diagnosis and treatment system, comprising:
an ultrasonic probe configured to transmit ultrasonic waves and receive echo signals;
an image generator unit configured to generate an ultrasonic image based on the echo signals received by said ultrasonic probe;
a biopsy needle configured to radiate radiofrequency waves; and
a controller unit configured to alternately perform the transmission of the ultrasonic wave by said ultrasonic probe and the radiofrequency wave radiation by said biopsy needle.

6. An ultrasonic diagnosis and treatment system according to claim 5, further comprising:
a radiofrequency wave cautery treatment device body coupled to said biopsy needle, wherein:
said controller unit comprises:
a transmission instruction unit configured to output a transmission instruction signal to a transmitter configured to provide a driving signal to said ultrasonic probe for transmitting the ultrasonic waves therefrom; and
a permission signal generator unit configured to generate a radiofrequency wave radiation permission signal for permitting the radiation of the radiofrequency waves from said biopsy needle, the output of the transmission instruction signal from said transmission instruction unit and the output of the radiofrequency wave radiation permission signal from said permission signal generator unit are alternately performed; and
said radiofrequency wave cautery treatment device body comprises:
a generator unit configured to generate a radiofrequency wave radiation instruction signal for radiating the radiofrequency waves from said biopsy needle; and
a radiofrequency wave radiation permission unit configured to output the radiofrequency wave radiation instruction signal to permit said biopsy needle to radiate the radiofrequency waves only when the radiofrequency wave radiation instruction signal is input from said radiofrequency wave radiation instruction signal generator unit and if the radiofrequency wave radiation permission signal is input from said permission signal generator unit.

7. An ultrasonic diagnosis and treatment system according to claim 6, wherein:
the radiofrequency wave radiation instruction signal from said radiofrequency wave radiation permission unit is output to a voltage generator unit configured to apply a voltage to said biopsy needle for radiating the radiofrequency waves.

8. An ultrasonic diagnosis and treatment system according to claim 5, further comprising:
an ultrasonic diagnosis apparatus comprising said ultrasonic probe, said image generator unit, and the controller unit; and
a radiofrequency wave cautery treatment device comprising said biopsy needle.

9. An ultrasonic diagnosis and treatment apparatus, comprising:
an ultrasonic probe configured to transmit ultrasonic waves and receive echo signals;
a biopsy needle configured to radiate radiofrequency waves;
a body coupled to said ultrasonic probe and said biopsy needle, said body comprising an image generator unit configured to generate an ultrasonic image based on the echo signals received by said ultrasonic probe, and a controller unit configured to alternately perform the transmission of the ultrasonic waves by the said ultrasonic probe and the radiofrequency wave radiation by said biopsy needle.

10. An ultrasonic diagnosis and treatment apparatus according to claim 9, wherein:
said controller unit further comprises:
a transmission instruction unit configured to output a transmission instruction signal to a transmitter unit configured to provide a driving signal to said ultrasonic probe to transmit the ultrasonic waves; and
a generator unit configured to generate a radiofrequency wave radiation instruction signal for radiating the radiofrequency waves from said biopsy needle, the output of the transmission instruction signal from said transmission instruction unit and the output of the radiofrequency wave radiation instruction signal from said radiofrequency wave radiation instruction signal generator unit are alternately performed.

11. An ultrasonic diagnosis and treatment apparatus according to claim 9, wherein:
said controller unit further comprises:
a transmission instruction unit configured to output a transmission instruction signal to a transmitter unit configured to provide a driving signal to said ultrasonic probe to transmit the ultrasonic waves; and
a permission signal generator unit configured to output a radiofrequency wave radiation permission signal to said biopsy needle, the output of the transmission instruction signal from said transmission instruction unit and the output of the radiofrequency wave radiation permission signal from said permission signal generator unit are alternately performed; and
said body further comprises a generator unit configured to generate a radiofrequency wave radiation instruction signal to said biopsy needle, said radiofrequency wave radiation permission unit is configured to output the radiofrequency wave radiation instruction signal to permit said biopsy needle to radiate the radiofrequency waves only when the radiofrequency wave radiation instruction signal is input from said radiofrequency wave radiation instruction signal generator unit and if the radiofrequency wave radiation permission signal from said permission signal generator unit is input.

12. An ultrasonic diagnosis apparatus, comprising:
an ultrasonic probe for configured to transmit ultrasonic waves and receive echo signals;
a radiofrequency wave cautery treatment device configured to radiate radiofrequency waves;
a transmission controller unit configured to control the transmission of the ultrasonic waves from said ultrasonic probe; and
an image generator unit configured to generate an ultrasonic image based on the echo signals received by said ultrasonic probe, wherein said transmission controller unit is configured to receive information with respect to the radiofrequency wave radiation from said radiofrequency wave cautery treatment device, and the transmission of ultrasonic waves from said ultrasonic probe is performed when the radiofrequency waves are not radiated from said radiofrequency wave cautery treatment device.

13. An ultrasonic diagnosis apparatus according to claim 12, wherein said radiofrequency wave, cautery treatment device comprises a biopsy needle configured to radiate the radiofrequency waves, said radiofrequency wave cautery treatment device is configured to output the information with respect to the radiofrequency wave radiation to said transmission controller.

14. An ultrasonic diagnosis and treatment system, comprising:
an ultrasonic probe configured to transmit ultrasonic waves and receive echo signals;
a transmission controller unit configured to control the transmission of the ultrasonic waves from said ultrasonic probe;
an image generator unit configured to generate an ultrasonic image based on the echo signals received by said ultrasonic probe;
a biopsy needle configured to radiate radiofrequency waves; and
a radiation controller unit configured to control the radiation of the radiofrequency waves from said biopsy needle, wherein said transmission controller unit is further configured to receive information with respect to the radiofrequency wave radiation from said radiation controller unit, and the ultrasonic waves transmission from said ultrasonic probe is performed when the radiofrequency wave is not radiated from said biopsy needle.

15. An ultrasonic diagnosis and treatment system according to claim 14, further comprising:
an ultrasonic diagnosis apparatus comprising said ultrasonic probe, said transmission controller unit, and said image generator unit, and
a radiofrequency wave cautery treatment device comprising said biopsy needle and said radiation controller unit.

16. An ultrasonic diagnosis apparatus according to claim 12, wherein said transmission controller unit comprises:
a transmission instructor unit configured to output a transmission instruction signal to a transmitter unit configured to provide to said ultrasonic probe a driving signal to transmit ultrasonic waves; and
a permission signal generator unit configured to generate a radiofrequency wave radiation permission signal that enables said radiofrequency wave cautery treatment device to radiate the radiofrequency waves.

17. An ultrasonic diagnosis apparatus according to claim 16, wherein said transmission controller unit further comprises:
a radiofrequency wave radiation permission unit configured to receive the radiofrequency wave radiation permission signal from said permission signal generator unit;
a generator unit configured to generate a radiofrequency wave radiation instruction signal for radiating the radiofrequency waves from said radiofrequency wave cautery treatment device, said radiofrequency wave radiation permission unit further configured to enable radiation of the radiofrequency waves from said radiofrequency wave cautery treatment device by outputting the radiofrequency wave radiation instruction signal only when the radiofrequency wave radiation instruction is input from said radiofrequency wave radiation instruction signal generator unit and if the radiofrequency wave radiation permission signal is input from said permission signal generator unit.

18. An ultrasonic diagnosis apparatus according to claim 12, wherein:
said radiofrequency wave cautery treatment device comprises a biopsy needle; and
said transmission controller unit further comprises:
a generator unit configured to generate radiofrequency wave radiation instruction signal for radiating the radiofrequency waves from said biopsy needle; and
a radiofrequency wave radiation permission unit configured to output the radiofrequency wave radiation instruction signal to radiate the radiofrequency waves from said biopsy needle only when the radiofrequency wave radiation instruction signal is input from said radiofrequency wave radiation instruction signal generator unit and if the radiofrequency wave radiation permission signal is input from said permission signal generator unit.

19. An ultrasonic diagnosis and treatment system according to claim 14, wherein said transmission controller unit comprises:
a transmission instructor unit configured to output a transmission instruction signal to a transmitter unit configured to provide to said ultrasonic probe a driving signal to transmit ultrasonic waves; and
a permission signal generator unit configured to generate a radiofrequency wave radiation permission signal that enables said biopsy needle to radiate the radiofrequency waves.

20. An ultrasonic diagnosis and treatment system according to claim 19, wherein said transmission controller unit further comprises:
a radiofrequency wave radiation permission unit configured to receive the radiofrequency wave radiation permission signal from said permission signal generator unit;
a generator unit configured to generate a radiofrequency wave radiation instruction signal for radiating the radiofrequency waves from said biopsy needle, said radiofrequency wave radiation permission unit further configured to enable radiation of the radiofrequency waves from said biopsy needle by outputting the radiofrequency wave radiation instruction signal only when the radiofrequency wave radiation instruction is input from said radiofrequency wave radiation instruction signal generator unit and if the radiofrequency wave radiation permission signal is input from said permission signal generator unit.
